(19) United States
(12) Patent Application Publication
(10) Pub. No.: US 2010/0015728 A1
(43) Pub. Date: Jan. 21, 2010

(54) Assay Device and Method

(75) Inventors: John William Dilleen, Clackmannanshire (GB); Phillip Lowe, Tullibody (GB); Andrew Gill, Alloa (GB)

Correspondence Address: Steptoe & Johnson LLP, 1330 Connecticut Avenue, NW, Washington DC, DC, 20036, US

(73) Assignee: IVERNESS MEDICAL SWITZERLAND GMBH, ZUG (CH)

(21) Appl. No.: 12/518,389

(22) PCT Filed: Dec. 19, 2007
(86) PCT No.: PCT/IB07/04022

§ 371 Date: Aug. 19, 2009

Related U.S. Application Data
(60) Provisional application No. 60/870,731, filed on Dec. 19, 2006.
(60) Provisional application No. 60/884,357, filed on Jan. 10, 2007.

(51) Int. Cl. G01N 33/536; B05D 5/12; B01J 8/00; G01N 27/26

(52) U.S. Cl. 436/536; 427/130; 422/68.1; 205/787

(57) ABSTRACT

A method for detecting an analyte can include binding an analyte with a first reagent which is associated with a magnetic particle, allowing analyte to interact with an excess amount of a second reagent capable of interacting with the analyte, and magnetically separating a portion of analyte-bound second reagent from excess second reagent. After the magnetic separation, the interaction of the analyte and the second reagent can be disrupted to produce a detectable form of the second reagent, which can be detected. A device and system suited to performing the method are also described.

### CLAIM FOR PRIORITY

This application claims priority under 35 U.S.C. §119(e) to U.S. Patent Application No. 60/870,731, filed on 19 Dec. 2006, and U.S. Patent Application No. 60/884,357, filed 10 Jan. 2007, each of which is hereby incorporated by reference in its entirety.

### TECHNICAL FIELD

The present invention relates to assay devices and methods

### BACKGROUND

Assays for species of interest (e.g., analytes) have many applications (e.g., in medicine, industry, and environmental analysis). For example, albumin is an analyte found in mammalian blood, a sample material that includes other components such as red blood cells, ionic species (e.g., various salts and metal ions), gases (e.g., solvated oxygen and nitrogen), and multiple biological compounds (e.g., proteins, lipoproteins, blood triglycerides, fatty acids and cholesterol). The metal-binding capacity of albumin can be related to a patient's health status.

### SUMMARY

Assays have been used to determine the presence of ischemia, a condition associated with poor oxygen supply to a part of the body due to, for example, a constriction or an obstruction of a blood vessel. Two common forms of ischemia include cardiovascular ischemia and cerebral ischemia. The former can be generally a direct consequence of coronary artery disease, while the latter often can be due to a narrowing of the arteries leading to the brain. When an ischemic event occurs, a portion of the subject's albumin (a blood protein) becomes modified. The modified albumin is referred to as to ischemia modified albumin (IMA). Determining the amount of IMA in the blood can be useful in the diagnosis of ischemic events.

One difference between IMA and normal albumin is that IMA has a lower capacity to bind certain metal ions. International Patent Publication WO 03/046538 describes electrochemical methods and a device for in vitro detection of an ischemic event in a patient sample. The publication describes adding a known amount of a transition metal ion to a sample and then measuring the current or potential difference of non-sequestered transition metal ion in the sample. The amount of non-sequestered transition metal ion in the sample reflects the degree of modification to albumin that is the result of an ischemic event. The WO 03/046538 publication is incorporated herein by reference in its entirety.

In one aspect, an assay device for detecting an analyte includes a channel configured to accept a liquid sample. The channel includes a magnetic particle, a first reagent capable of selectively binding the analyte and capable of linking to the magnetic particle, a second reagent capable of interacting with the analyte, and a third reagent capable of disrupting the interaction of the analyte and the second reagent, thereby producing a detectable form of the second reagent.

In another aspect, a method of detecting an analyte in a liquid sample includes associating a magnetic particle with a first reagent capable of selectively binding the analyte, binding the analyte with the first reagent, allowing analyte to interact with an excess amount of a second reagent capable of interacting with the analyte, magnetically separating a portion of analyte-bound second reagent from excess second reagent, disrupting the interaction of the analyte and the second reagent, thereby producing a detectable form of the second reagent; and detecting the detectable form of the second reagent.

In another aspect, a method of detecting an analyte in a liquid sample includes introducing the liquid sample into an assay device for detecting the analyte, the device including a channel configured to accept the liquid sample, and detecting the detectable form of the second reagent. The channel can include a magnetic particle, a first reagent capable of selectively binding the analyte and capable of linking to the magnetic particle, a second reagent capable of interacting with the analyte, and a third reagent capable of disrupting the interaction of the analyte and the second reagent, thereby producing a detectable form of the second reagent.

In another aspect, a system for detecting an analyte including an assay device includes an assay device including a channel configured to accept a liquid sample and an assay device operator configured to detect the detectable form of the second reagent. The channel can include a magnetic particle, a first reagent capable of selectively binding the analyte and capable of linking to the magnetic particle, a second reagent capable of interacting with the analyte, and a third reagent capable of disrupting the interaction of the analyte and the second reagent, thereby producing a detectable form of the second reagent. The device operator can be further configured to determine a value describing the concentration of the analyte in the liquid sample. Alternatively or in addition, the device operator can be further configured to correlate the value with a health status of a patient.

In another aspect, a method of making a device includes depositing a magnetic particle on a substrate, depositing a first reagent on the substrate, the first reagent being capable of selectively binding an analyte and capable of linking to the magnetic particle, depositing a second reagent on the substrate, the second reagent being capable of interacting with the analyte, and depositing a third reagent on the substrate, the third reagent being capable of disrupting the interaction of the analyte and the second reagent.

The first reagent can include an anti-human serum albumin antibody. The second reagent can include a first divalent metal ion, for example, Co²⁺. The third reagent can include a second divalent metal ion, for example, Ni²⁺. The third reagent can be capable of interacting with the analyte or the second reagent.

The third reagent can be capable of forming a complex with the second reagent. The third reagent can be a ligand for the first divalent metal ion.

In the method, the detectable form of the reagent can be detected by electrochemical detection, optical detection, or combinations thereof.

The assay method and device can be used in home testing kits for analyzing species present in the blood. In particular, the device and method facilitate the performance of more than one assay on a small sample volume, and are suitable for use with home testing kits that use the “finger stick” or “finger prick” procedure. The assay device and method can accept small fluid samples in a simple step, and is able to present small fluid samples for immediate testing in a reliable and reproducible fashion.

Other features, objects, and advantages will be apparent from the description, the drawings and the claims.

### BRIEF DESCRIPTION OF THE DRAWINGS

FIG. 1A is a schematic top view of an assay device. FIGS. 1B-1E are schematic side views of the device at different stages of operation.

FIG. 2 is a schematic drawing of a device operator.

### DETAILED DESCRIPTION

One component of blood is human serum albumin (HSA). Exposure of HSA to ischemic tissue produces modifications to the N-terminus, and possibly other sites, on the albumin molecule. The N-terminus of albumin has been well characterized as being the primary binding site for several transition metals such as cobalt, nickel and copper. This altered albumin is referred to as Ischemia Modified Albumin (IMA). Therefore, if a known amount of a transition metal is added to a biological sample (for example, a patient sample of whole blood, serum or plasma, urine, cerebrospinal fluid, or saliva), normal albumin can be distinguished from IMA on the basis of differential metal binding. Metal added to the sample will be bound to a greater extent in a non-ischemic sample than in an ischemic sample, wherein the conversion of albumin to IMA reduces the metal binding capacity of the sample. The unbound metal can then be detected and quantified, for example by using the Albumin Cobalt Binding (ACB) Test (Ischemia Technologies, Inc., Denver, Colo.), which uses colorimetric methods to determine the amount of IMA present in the sample. See generally WO 03/046538, WO 00/20840, and U.S. Pat. No. 5,227,307, each of which is incorporated by reference in its entirety.

Generally, an assay device can include a reagent that can selectively bind an analyte that is present (or potentially present) in a sample. In some circumstances, the analyte-binding reagent is linked to a magnetic particle to facilitate a magnetic separation of the bound analyte from other, unbound components of the sample. Such separation can be advantageous for assay sensitivity and reproducibility.

In some cases, the analyte is further capable of binding a detectable species. The capacity of the analyte to bind the detectable species can be related to a patient's health status. The detectable species can be supplied in excess to the analyte's binding capacity prior to a magnetic separation. After the separation, the detectable species can be released from the analyte and detected. In this way, the assay can directly measure the analyte's binding capacity for the detectable species.

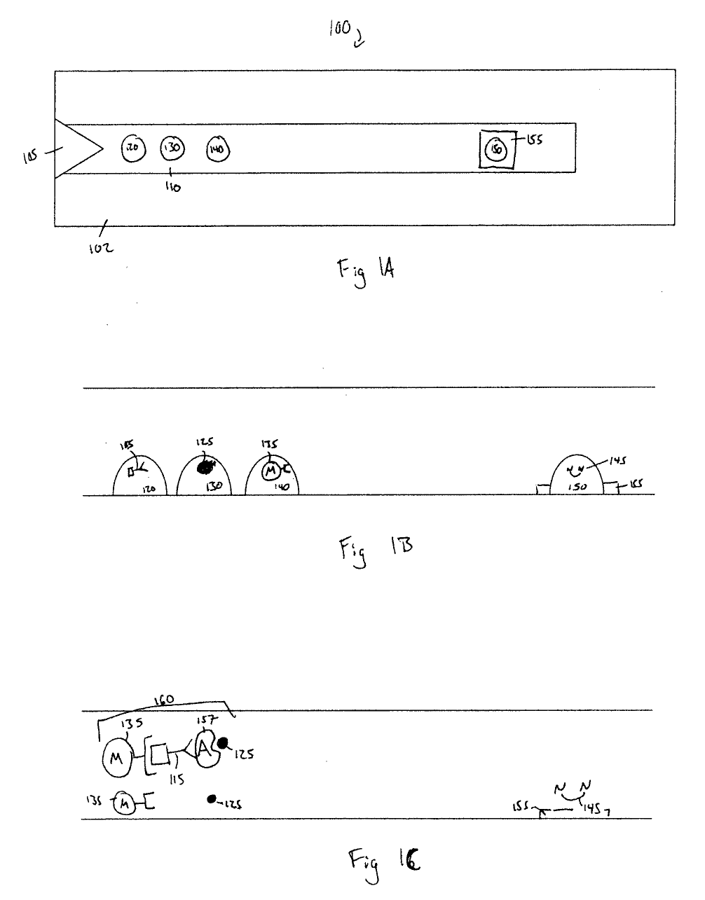

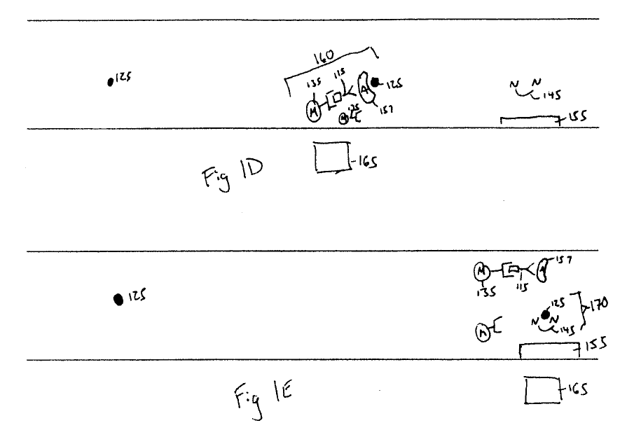

Referring to FIG. 1A, an assay device 100 includes a sample inlet 105 fluidly connected to flow channel 110. Flow channel 110 includes a first reagent 115 in first reagent zone 120, a second reagent 125 in second reagent zone 130, magnetic particles 135 in particle zone 140, a third reagent 145 in third reagent zone 150 and detection zone 155. Flow channel 110 can optionally include a reference zone. A reagent zone includes one or more reagents in a dry state on a surface of channel 110. In FIG. 1, the absolute and relative locations of reagent zones 120, 130, and 150, and of particle zone 140 and detection zone 155, are illustrative only and not meant to be limiting.

Device 100 can include base 102 covered by a lid, which seals against a surface of base 102. The seal between base 102 and lid ensures that channel 110 is liquid-tight. The lid can include through holes at appropriate locations, e.g., at inlet 105. The can be sealed to base 102 by an adhesive layer. The adhesive layer can be, for example, a heat activated adhesive or a pressure activated adhesive.

First reagent 115 is capable of selectively binding to a desired component, or analyte 157, in a sample fluid. For example, first reagent 115 can be capable of selectively binding human serum albumin, which is present in sample fluids including human blood [and human blood plasma]. In some embodiments, reagent 115 is capable of binding a modified form of the analyte with substantially similar affinity as the unmodified form. For example, the first reagent can selectively bind human serum albumin in either its unmodified form or its ischemia-modified form (IMA) with substantially similar affinity. First reagent 115 is present in flow channel 110 in an amount in excess of the amount of analyte expected in a sample fluid, so that substantially all of the analyte will become bound to first reagent 115. The first reagent can be an anti-human serum albumin antibody, for example, monoclonal antibody 4N91/91, Biogenesis catalog no. 0220-0704.

The magnetic particle 135 can be directly linked or indirectly linked to the first reagent 115. A direct link can include, for example, a covalent bond, as in the case of antibody linked to a magnetic particle by an amide bond or other covalent bond. A direct link can also include links where the first reagent 115 is separated from the magnetic particle by a spacer, but the overall linkage is covalent in nature. An indirect link can include a non-covalent affinity interaction. Non-covalent affinity interactions occur between complementary chemical moieties, such as between biotin and avidin or streptavidin; between FK506 and FK506 binding protein; between single strands of nucleic acids having complementary base sequences; and the like. Thus, the magnetic particle 135 can be directly linked to one member of a non-covalent affinity interaction, and the first reagent 115 can be directly linked the other member of the non-covalent affinity interaction. When the non-covalent affinity interaction is formed between the two members, the first reagent 115 becomes linked indirectly to the magnetic particle 135. Preferably, the non-covalent affinity interaction is one of high affinity and high specificity; in other words, once the non-covalent affinity interaction is formed, the member remain substantially bound to one another, and the members do not substantially bind to other components of a sample liquid.

A magnetic particle is a particle that is influenced by a magnetic field. The magnetic particle can be, for example, a magnetic particle described, in U.S. Patent Application Publication Nos. 20050147963 or 20050100930, or U.S. Pat. No. 5,348,876, each of which is incorporated by reference in its entirety, or commercially available beads, for example, those produced by Dynal AS under the trade name DYNABEADS™. In particular, antibodies linked to magnetic particles are described in, for example, United States Patent Application Nos. 20050149169, 20050148096, 20050142549, 20050074748, 20050148096, 20050106652, and 20050100930, and U.S. Pat. No. 5,348,876, each of which is incorporated by reference in its entirety.

Second reagent 125 is capable of interacting with the same analyte as does first reagent 115. In particular, second reagent 125 is capable of binding to the analyte simultaneously with first reagent 115. The second reagent may interact differentially with different forms of the analyte. For example, the interaction between the analyte and the second reagent may be stronger for one form of the analyte than for another form. This differential interaction can be useful in determining the relative quantities of different forms of the analyte. Second reagent 125 is present in flow channel 110 in an amount in excess of the amount of analyte expected in a sample fluid, so that substantially all of the analyte will become bound to second reagent 125. The second reagent can be a metal ion capable of binding to human serum albumin, such as, for example, a divalent transition metal ion, for example, Co²⁺, Cu²⁺, or Ni²⁺.

The device can be configured (e.g., with respect to the location of reagent and particle zones) such that when a sample fluid is introduced, a magnetic particle-first gent-analyte-second reagent complex 160 is formed. Complex 160 is preferably formed a distance away from detection zone 155. It can be desirable to separate complex 160 from other components in the fluid (e.g., from other sample components that can interfere with detection, or from quantities of second reagent 125 in excess to the capacity of the analyte to bind second reagent 125). An applied magnetic field can be used to move magnetic particles 135 (and all that is bound to them) without carrying along other materials not bound to the magnetic particles. In this way, complex 160 can be separated from other components in the fluid, and can be brought to third reagent zone 150.

Third reagent 145 is capable of disrupting the complex formed between second reagent 125 and the analyte to produce a detectable form 170 of second reagent 125. The detectable form of second reagent 125 can be, for example, unbound (i.e., free) second reagent 125, or a complex of second reagent 125 with a ligand. The detectable form 170 of second reagent 125 can be detected by, for example, optical or electrochemical detection.

In one embodiment, the analyte-second reagent complex can be disrupted when the third reagent 145 displaces second reagent 125 from the analyte (forming a third reagent-analyte complex), while second reagent 125 becomes unbound (free). Free second reagent 125 can be detected at detection zone 155. For example, when the analyte is human serum albumin and the second reagent is Co²⁺, the third reagent can be Ni²⁺. The third reagent can be provided in excess to the amount of analyte-second reagent complex, in order to promote the complete disruption of the analyte-second reagent complex, and thus to ensure that all of the second reagent that was present in the analyte-second reagent complex is freed.

In another embodiment, the analyte-second reagent complex can be disrupted when third reagent 145 displaces the analyte from second reagent 125 (forming a third reagent-second reagent complex). Preferably, third reagent 145 has a substantially higher affinity for second reagent 125 than does first reagent 115. As such, third reagent 145 will displace first reagent 115 from second reagent 125. When the second reagent is a metal ion, the third reagent can be a ligand for the metal ion, such as a chelating ligand having a higher affinity for the metal ion than does human serum albumin (whether unmodified or ischemia-modified). In particular, the ligand can be chosen based on the ease of detection of the third reagent-second reagent complex. When the second reagent is Co²⁺, one ligand suitable for use as third reagent 145 is 1,10-phenanthroline, which forms an electrochemically detectable complex with Co²⁺. Another species suitable for third reagent 145 is dithiothreitol, which forms an optically detectable complex with Co²⁺.

Reagents 115, 125, 145, and magnetic particles 135 can each be provided in a dry form on a surface in fluid channel 110. When a sample fluid encounters a reagent or particle in dry form (as when the fluid travels along channel 110 via capillary action) the reagent or particle can become resuspended and/or dissolved in the sample fluid.

In some embodiments, the detection zone can include one or more electrodes. The electrodes can be formed of a material selected for electrical conductivity and low reactivity with sample components, for example, silver, gold, aluminum, palladium, platinum, iridium, a conductive carbon, a doped tin oxide, stainless steel, or a conductive polymer. The electrodes in the detection zones can measure an electrical property of the sample, such as a voltage or a current.

Alternatively, the detection zones and the reference zones can each have at least one working electrode and counter electrode. That is, the detection and reference zones can make independent measurements. Optionally, counter electrodes are also included in the assay device. Assay devices including electrodes for measuring electrical properties of a sample are described in, for example, U.S. Pat. Nos. 5,708,247, 6,241,862, and 6,733,655, each of which is incorporated by reference in its entirety.

The electrodes can be formed of a material selected for electrical conductivity and low reactivity with sample components, for example, silver, gold, aluminum, palladium, platinum, iridium, a conductive carbon, a doped tin oxide, stainless steel, or a conductive polymer. The electrodes can measure an electrical property of the sample, such as a voltage or a current. Assay devices including electrodes for measuring electrical properties of a sample are described in, for example, U.S. Pat. Nos. 5,708,247, 6,241,862, and 6,733,655, each of which is incorporated by reference in its entirety.

In some embodiments, the assay device base, assay device lid, or both have a translucent or transparent window aligned with the detection zone. An optical change that occurs in the detection zone can be detected through the window. Detection can be done visually (i.e., the change is measured by the user's eye) or measured by an instrument (e.g., a photodiode, photomultiplier, or the like). In general, the reference zone is similar in nature to the detection zone. In other words, when the detection zone includes an electrode, the reference can likewise include an electrode. When the detection zone is aligned with a window for optical measurement, the reference zone can similarly be aligned with a window for optical measurement. In some embodiments, the reference zone is not adapted to collect analyte. Alternatively, the reference zone is adapted to collect analyte, but performs a different analysis on said analyte. Thus, the detectable change measured in the reference zone can be considered a background measurement to be accounted for when determining the amount of analyte present in the sample.

A description of the operation of the device follows with reference to FIGS. 1B-1E. For the purposes of illustration, the description is given for a device configured to determine electrochemically the cobalt-binding capacity of albumin in a sample of blood, using a chelating ligand (e.g., 1,10-phenanthroline) as the third reagent. This illustration is not limiting. FIG. 1B shows the assay device in the dry, as-provided state. A sample of blood enters fluid channel 110 via inlet 105. The blood travels from inlet 105 along channel 110 via capillary action, resuspending the dry reagents 115, 125, 135 and 145 (anti-albumin antibody linked to biotin, CoCl₂, magnetic particles linked to streptavidin, and 1,10-phenanthroline, respectively). FIG. 1C shows that as the reagents become resuspended, complexes between those components with mutual affinity are formed. More specifically, Co²⁺ binds to the albumin in the blood. Because Co²⁺ is present in excess to the Co²⁺ binding capacity of the albumin, substantially all of the Co²⁺ binding sites on the albumin are bound to albumin. Put another way, the albumin Co²⁺ binding sites are saturated. Simultaneously, the anti-albumin antibodies bind to albumin. Similarly, an excess of antibody ensures that substantially all of the albumin in the sample is bound to antibody. In this way, substantially all of the albumin in the sample becomes bound in an antibody-albumin-Co²⁺ complex. The biotin-streptavidin interaction further links the antibodies to magnetic particles.

A magnetic field is then applied by a magnetic field source 165. The magnetic field source can be configured to provide a shaped magnetic field. A shaped magnetic field can have magnetic field lines designed to direct magnetic particles toward a desired location, such as the detection zone 155. Such a shaped magnetic field can be useful to control the diffusion or migration of magnetic particles. More than one magnetic field source can be provided, particularly when a shaped magnetic field is desired. For example, magnetic field sources can be provided at either end of an assay device, where one is configured to attract magnetic particles and the other to repel magnetic particles. Such a configuration can favor the location of all magnetic particles a desired location in channel 110.

The applied magnetic field can drive the magnetic particles, and by extension, substantially all of the albumin-cobalt complexes to a desired location along the flow channel. FIG. 1D illustrates how source 165 can drive complex 160 and excess particles 135 from their initial location towards detection zone 155. Only the portion of second reagent 125 that is part of complex 160 is carried along towards detection zone 155; excess unbound second reagent 125 is left behind.

An optional wash step can displace blood in the flow channel with a desired buffer. A buffer pouch incorporated into the device can deliver a wash buffer, and the composition of the buffer can be varied (e.g., sodium acetate, phosphate-citrate, sodium citrate or any other buffer at any suitable concentration or pH). Any suitable liquid can be used instead of a buffer (see, for example, U.S. Patent Application No. 60/736,302, filed Nov. 15, 2005, which is incorporated by reference in its entirety).

The applied magnetic field is then manipulated (e.g., by moving a permanent magnet relative to the flow channel) to move the magnetic particles. Excess cobalt (i.e., cobalt not bound to albumin) does not travel with the magnetic particles. The magnetic particles and associated material (including albumin and Co²⁺ bound to albumin) are moved to the detection zone, where the third reagent 145, 1,10-phenanthroline (phen), is present. See FIG. 1E. The phen is present in a quantity sufficient to ensure that substantially all of the Co²⁺ bound to albumin is instead bound to phen. This is facilitated by the choice of phen as the third reagent: the relative stabilities of Co²⁺-phen and Co²⁺-albumin complexes are such that the equilibrium will lie greatly in favor of Co²⁺-phen complexes. As such, Co²⁺-phen complexes (i.e., Co(phen)₃²⁺) are formed in an amount substantially equal to the Co²⁺ binding capacity of the albumin in the sample. The Co(phen)₃²⁺ are the detectable form 170 of second reagent 125.

At this point, the electrodes in the detection zone are activated (e.g., by an operator device configured to do so) to electrochemically detect the Co(phen)₃²⁺ complexes. These complexes can be electrochemically detected at more moderate voltages than can free Co²⁺, and the detection does not result in the plating of Co⁰ (i.e., cobalt metal) on an electrode. Furthermore, the electrochemical signal of Co(phen)₃²⁺ can be distinguished from other electrochemical signals that can arise from other sample components. As such, electrochemical detection of Co(phen)₃²⁺ can be more reliable than detection of other species (e.g., free cobalt) in terms of confidence that the signal detected arises from the species to be determined. Additionally, the amount detected Co(phen)₃²⁺ is directly related to the cobalt binding capacity of albumin in the sample. Other albumin cobalt binding assays can measure the amount of free cobalt remaining after albumin binds a portion of a known amount of cobalt. In that configuration, the measured amount of cobalt is indirectly related to the albumin binding capacity (which in turn is related to the health status of the patient) of the sample.

The analyte can be a biomarker for a condition that afflicts the mammalian body. The term “biomarker” refers to a biochemical in the body that has a particular molecular trait to make it useful for diagnosing a condition, disorder, or disease and for measuring or indicating the effects or progress of a condition, disorder, or disease. For example, common biomarkers found in a person's bodily fluids (i.e., breath or blood), and the respective diagnostic conditions of the person providing such biomarkers include, but are not limited to, ischemia modified albumin “IMA” (source: lack of oxygen to the blood; diagnosis: coronary artery disease), N-terminal truncated pro-brain natriuretic peptide “NT pro-BNP” (source: stretching of myocytes; diagnosis: congestive heart failure), acetaldehyde (source: ethanol; diagnosis: intoxication), acetone (source: acetoacetate; diagnosis: diet; ketogenic/diabetes), ammonia (source: deamination of amino acids; diagnosis: uremia and liver disease), CO (carbon monoxide) (source: CH₂Cl₂, elevated % COH; diagnosis: indoor air pollution), chloroform (source: halogenated compounds), dichlorobenzene (source: halogenated compounds), diethylamine (source: choline; diagnosis: intestinal bacterial overgrowth), H₂ (hydrogen) (source: intestines; diagnosis; lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source; metabolism; diagnosis: periodontal disease/ovulation), MeS (source: metabolism; diagnosis: cirrhosis), and Me₂S (source: infection; diagnosis: trench mouth).

The sample can be any biological fluid, such as, for example, blood, blood plasma, serum, urine, saliva, mucous, tears, or other bodily fluid. The sample could also be an environmental sample. The analyte can be any component that is found (or may potentially be found) in the sample, such as, for example, a protein, a peptide, a nucleic acid, a metabolite, a saccharide or polysaccharide, a lipid, a drug or drug metabolite, or other component. The assay device can optionally be supplied with a blood separation membrane arranged between a sample inlet and the detection zone, such that when whole blood is available as a sample, only blood plasma reaches the detection zone.

The assay device and included reagents are typically provided in a dry state. Addition of a liquid sample to the assay device (i.e., to the capillary channel) can resuspend dry reagents.

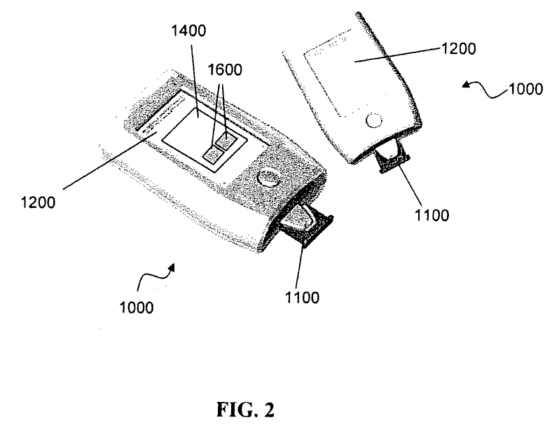

Referring to FIG. 2, reader instrument 1000 accepts test assay device 1100 and includes display 1200. The display 1200 may be used to display images in various formats, for example, text, joint photographic experts group (JPEG) format, tagged image file format (TIFF), graphics interchange format (GIF), or bitmap. Display 1200 can also be used to display text messages, help messages, instructions, queries, test results, and various information to patients. Display 1200 can provide a user with an input region 1400. Input region 1400 can include keys 1600. In one embodiment, input region 1400 can be implemented as symbols displayed on the display 1200, for example when display 1200 is a touch-sensitive screen. User instructions and queries are presented to the user on display 1200. The user can respond to the queries via the input region.

Reader 1000 also includes an assay device reader, which accepts diagnostic test assay devices 1100 for reading. The assay device reader can measure the level of an analyte based on, for example, the magnitude of an optical change, an electrical change, or other detectable change that occurs on a test assay device 1100. For reading assay devices that produce an optical change in response to analyte, the assay device reader can include optical systems for measuring the detectable change, for example, a light source, filter, and photon detector, e.g., a photodiode, photomultiplier, or Avalance photo diode. For reading assay devices that produce an electrical change in response to analyte, the assay device reader can include electrical systems for measuring the detectable change, including, for example, a voltameter or amperometer.

Device 1000 further can include a communication port (not pictured). The communication port can be, for example, a connection to a telephone line or computer network. Device 1000 can communicate the results of a measurement to an output device, remote computer, or to a health care provider from a remote location. A patient, health care provider, or other user can use reader 1000 for testing and recording the levels of various analytes, such as, for example, a biomarker, a metabolite, or a drug of abuse.

Various implementations of diagnostic device 1000 may access programs and/or data stored on a storage medium (e.g., a hard disk drive (HDD), flash memory, video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the reader can be in the form of a software application running on any processing device, such as, a general-purpose computing device, a personal digital assistant (PDA), a special-purpose computing device, a laptop computer, a handheld computer, or a network appliance. The reader may be implemented using a hardware configuration including a processor, one or more input devices, one or more output devices, a computer-readable medium, and a computer memory device. The processor may be implemented using any computer processing device, such as, a general-purpose microprocessor or an application specific integrated circuit (ASIC).

The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter. The display 1200 may be implemented using any output technology, including a liquid crystal display (LCD), a television, a printer, and a light emitting diode (LED).

The computer-readable medium provides a mechanism for storing programs and data either on a fixed or removable medium. The computer-readable medium may be implemented using a conventional computer hard drive, or other removable medium. Finally, the system uses a computer memory device, such as a random access memory (RAM), to assist in operating the reader. Implementations of the reader can include software that directs the user in using the device, stores the results of measurements. The reader 1000 can provide access to applications such as a medical records database or other systems used in the care of patients. In one example, the device connects to a medical records database via the communication port. Device 1000 may also have the ability to go online, integrating existing databases and linking other websites.

In general, the assay device can be made by depositing reagents on a base and sealing a lid over the base. The base can be a micro-molded platform or a laminate platform.

Micro-Molded Platform

For an assay device prepared for optical detection, the base, the lid, or both base and lid can be transparent to a desired wavelength of light. Typically both base and lid are transparent to visible wavelengths of light, e.g., 400-700 nm. The base and lid can be transparent to near UV and near IR wavelengths, for example, to provide a range of wavelengths that can be used for detection, such as 200 nm to 1000 nm, or 300 nm to 900 nm.

For an assay device that will use electrochemical detection, electrodes are deposited on a surface of the base. The electrodes can be deposited by screen printing on the base with a carbon or silver ink, followed by an insulation ink; by evaporation or sputtering of a conductive material (such as, for example, gold, silver or aluminum) on the base, followed by laser ablation; or evaporation or sputtering of a conductive material (such as, for example, gold, silver or aluminum) on the base, followed by photolithographic masking and a wet or dry etch.

An electrode can be formed on the lid in one of two ways. A rigid lid can be prepared with one or more through holes, mounted to a vacuum base, and screen-printing used to deposit carbon or silver ink. Drawing a vacuum on the underside of the rigid lid while screen printing draws the conductive ink into the through holes, creating electrical contact between the topside and underside of the lid, and sealing the hole to ensure that no liquid can leak out.

Alternatively, the lid can be manufactured without any through holes and placed, inverted, on a screen-printing platform, where carbon or silver ink is printed. Once the electrodes have been prepared, the micro-molded bases are loaded and registered to a known location for reagent deposition. Deposition of reagents can be accomplished by dispensing or aspirating from a nozzle, using an electromagnetic valve and servo- or stepper-driven syringe. These methods can deposit droplets or lines of reagents in a contact or non-contact mode. Other methods for depositing reagents include pad printing, screen printing, piezoelectric print head (e.g., ink-jet printing), or depositing from a pouch which is compressed to release reagent (a “cake icer”). Deposition can preferably be performed in a humidity- and temperature-controlled environment. Different reagents can be dispensed at the same or at a different station. Fluorescent or colored additives can optionally be added to the reagents to allow detection of cross contamination or overspill of the reagents outside the desired deposition zone. Product performance can be impaired by cross-contamination. Deposition zones can be in close proximity or a distance apart. The fluorescent or colored additives are selected so as not to interfere with the operation of the assay device, particularly with detection of the analyte.

After deposition, the reagents are dried. Drying can be achieved by ambient air-drying, infrared drying, infrared drying assisted by forced air, ultraviolet light drying, forced warm, controlled relative humidity drying, or a combination of these. Micro-molded bases can then be lidded by bonding a flexible or rigid lid on top. Registration of the base and lid occurs before the two are bonded together. The base and lid can be bonded by heat sealing (using a heat activated adhesive previously applied to lid or base, by ultrasonic welding to join two similar materials, by laser welding (mask or line laser to join two similar materials), by cyanoacrylate adhesive, by epoxy adhesive previously applied to the lid or base, or by a pressure sensitive adhesive previously applied to the lid or base. After lidding, some or all of the assembled assay devices can be inspected for critical dimensions, to ensure that the assay device will perform as designed. Inspection can include visual inspection, laser inspection, contact measurement, or a combination of these.

The assay device can include a buffer pouch. The buffer pouch can be a molded well having a bottom and a top opening. The lower opening can be sealed with a rupturable foil or plastic, and the well filled with buffer. A stronger foil or laminate is then sealed over the top opening. Alternatively, a preformed blister pouch filled with buffer is placed in and bonded in the well. The blister pouch can include 50 to 200 μL of buffer and is formed, filled, and sealed using standard blister methods. The blister material can be foil or plastic. The blister can be bonded to the well with pressure sensitive adhesive or a cyanoacrylate adhesive.

Laminate Platform

Three or more laminates, fed on a roll form at a specified width, can be used to construct an assay device. The base laminate is a plastic material and is coated on one surface with a hydrophilic material. This laminate is fed into a printing station for deposition of conductive electrodes and insulation inks. The base laminate is registered (cross web) and the conductive electrodes deposited on the hydrophilic surface, by the techniques described previously. The base laminate is then fed to a deposition station and one or more reagents applied to the laminate. Registration, both cross web and down web, occurs before reagents are deposited by the methods described above. The reagents are dried following deposition by the methods described above. A middle laminate is fed in roll form at a specified width. There can be more than one middle laminate in an assay device. The term middle serves to indicate that it is not a base laminate or lid laminate. A middle laminate can be a plastic spacer with either a pressure sensitive adhesive or a heat seal adhesive on either face of the laminate. A pressure sensitive adhesive is provided with a protective liner on either side to protect the adhesive. Variations in the thickness of the middle laminate and its adhesives are less than 15%, or less than 10%.

Channels and features are cut into the middle laminate using a laser source (e.g., a CO₂ laser, a YAG laser, an excimer laser, or other). Channels and features can be cut all the way through the thickness of the middle laminate, or the features and channels can be ablated to a controlled depth from one face of the laminate. The middle and base laminates are registered in both the cross web and down web directions, and bonded together. If a pressure sensitive adhesive is used, the lower liner is removed from the middle laminate and pressure is applied to bond the base to the middle laminate. If a heat seal adhesive is used, the base and middle laminate are bonded using heat and pressure.

The top laminate, which forms the lid of the assay device, is fed in roll form at a specified width. The top laminate can be a plastic material. Features can be cut into the top laminate using a laser source as described above. The top laminate is registered (cross web and down web) to the base and middle laminates, and bonded by pressure lamination or by heat and pressure lamination, depending on the adhesive used. After the laminate is registered in cross and down web directions, discrete assay devices or test strips are cut from the laminate using a high powered laser (such as, for example, a CO₂ laser, a YAG laser, an excimer laser, or other).

Some, or all, of the assembled assay devices can be inspected for critical dimensions, to ensure that the assay device will fit perform as designed. Inspection can include visual inspection, laser inspection, contact measurement, or a combination of these.

An example of one application that employs the use of assays to detect analytes is the analysis of physiological fluid samples, such as blood samples. In particular, it has become increasingly common to analyse blood samples for analytes that may be indicative of disease or illness. Such analyses can be performed using an assay that directly or indirectly detects an analyte of interest.

Other embodiments are within the scope of the following claims.

What is claimed is:

1. An assay device for detecting an analyte comprising:
a channel configured to accept a liquid sample, the channel including:
a magnetic particle;
a first reagent capable of selectively binding the analyte and capable of linking to the magnetic particle;
a second reagent capable of interacting with the analyte; and
a third reagent capable of disrupting the interaction of the analyte and the second reagent, thereby producing a detectable form of the second reagent.

2. The device of claim 1, wherein the first reagent includes an anti-human serum albumin antibody.

3. The device of claim 2, wherein the second reagent includes a first divalent metal ion.

4. The device of claim 3, wherein the first divalent metal ion is Co²⁺.

5. The device of claim 4, wherein the third reagent includes a second divalent metal ion.

6. The device of claim 5, wherein the second divalent metal ion is Ni²⁺.

7. The device of claim 3, wherein the third reagent is capable of forming a complex with the second reagent.

8. The device of claim 7, wherein the third reagent is a ligand for the first divalent metal ion.

9. A method of detecting an analyte in a liquid sample, comprising:
associating a magnetic particle with a first reagent capable of selectively binding the analyte;
binding the analyte with the first reagent;
allowing analyte to interact with an excess amount of a second reagent capable of interacting with the analyte;
magnetically separating a portion of analyte-bound second reagent from excess second reagent;
disrupting the interaction of the analyte and the second reagent, thereby producing a detectable form of the second reagent; and
detecting the detectable form of the second reagent.

10. The method of claim 9, wherein the first reagent includes an anti-human serum albumin antibody.

11. The method of claim 10, wherein the second reagent includes a first divalent metal ion.

12. The method of claim 11, wherein the first divalent metal ion is Co²⁺.

13. The method of claim 12, wherein the third reagent includes a second divalent metal ion.

14. The method of claim 13, wherein the second divalent metal ion is Ni²⁺.

15. The method of claim 11, wherein the third reagent is capable of forming a complex with the second reagent.

16. The method of claim 15, wherein the third reagent is a ligand for the first divalent metal ion.

17. The method of claim 9, wherein detecting the detectable form of the second reagent includes electrochemical detection.

18. The method of claim 9, wherein detecting the detectable form of the second reagent includes optical detection.

19. A method of detecting an analyte in a liquid sample, comprising:
introducing the liquid sample into an assay device for detecting the analyte, the device including a channel configured to accept the liquid sample, the channel including:
a magnetic particle;
a first reagent capable of selectively binding the analyte and capable of linking to the magnetic particle;
a second reagent capable of interacting with the analyte; and
a third reagent capable of disrupting the interaction of the analyte and the second reagent, thereby producing a detectable form of the second reagent; and
detecting the detectable form of the second reagent.

20. The method of claim 19, further comprising detecting the detectable form of the second reagent.

21. The method of claim 20, wherein the detectable form of the second reagent includes electrochemical detection.

22. The method of claim 20, wherein detecting the detectable form of the second reagent includes optical detection.

23. A system for detecting an analyte comprising an assay device including
an assay device including a channel configured to accept a liquid sample, the channel including:
a magnetic particle;
a first reagent capable of selectively binding the analyte and capable of linking to the magnetic particle;
a second reagent capable of interacting with the analyte; and
a third reagent capable of disrupting the interaction of the analyte and the second reagent, thereby producing a detectable form of the second reagent; and
an assay device operator configured to detect the detectable form of the second reagent.

24. The system of claim 23, wherein the device operator is further configured to determine a value describing the concentration of the analyte in the liquid sample.

25. The system of claim 23, wherein the device operator is further configured to correlate the value with a health status of a patient.

26. A method of making a device comprising:
depositing a magnetic particle on a substrate;
depositing a first reagent on the substrate, the first reagent being capable of selectively binding an analyte and capable of linking to the magnetic particle;
depositing a second reagent on the substrate, the second reagent being capable of interacting with the analyte; and
depositing a third reagent on the substrate, the third reagent being capable of disrupting the interaction of the analyte and the second reagent.

27. The method of claim 26, wherein the third reagent is capable of interacting with the analyte.

28. The method of claim 26, wherein the third reagent is capable of interacting with the second reagent.

29. The method of claim 26, wherein the first reagent includes an anti-human serum albumin antibody.

30. The method of claim 29, wherein the second reagent includes a first divalent metal ion.

31. The method of claim 30, wherein the first divalent metal ion is Co²⁺.

32. The method of claim 31, wherein the third reagent includes a second divalent metal ion.

33. The method of claim 32, wherein the second divalent metal ion is Ni²⁺.

34. The method of claim 30, wherein the third reagent is capable of forming a complex with the second reagent.

35. The method of claim 34, wherein the third reagent is a ligand for the first divalent metal ion.
